(19) United States
(12) Patent Application Publication
(10) Pub. No.: US 2026/0102369 A1
(43) Pub. Date: Apr. 16, 2026

(54) COMPOSITION AND METHOD FOR TREATING MUSCLE DECLINE ASSOCIATED WITH RENAL DISEASE OR DYSFUNCTION

(71) Applicant: SOCIÉTÉ DES PRODUITS NESTLÉ S.A., Vevey (CH)

(72) Inventors: Vincenzo SORRENTINO, Lausanne (CH); Sonia KARAZ, Epalinges (CH); Yuanlong PAN, Chesterfield, MO (US); Qinghong LI, Chesterfield, MO (US)

(21) Appl. No.: 19/114,929

(22) PCT Filed: Sep. 28, 2023
(86) PCT No.: PCT/EP2023/076833

§ 371 Date: Mar. 25, 2025

(30) Foreign Application Priority Data
Sep. 30, 2022 (EP) 22199020.3

(51) Int. Cl. A61K 31/23; A61K 31/197; A61K 31/198; A61K 31/355; A61K 31/375; A61K 31/4188; A61K 31/4406; A61K 31/4415; A61K 31/51; A61K 31/519; A61K 31/525; A61K 31/714; A61K 35/60; A61P 13/12; A61P 21/00

(52) U.S. Cl. CPC A61K 31/23; A61K 31/197; A61K 31/198; A61K 31/355; A61K 31/375; A61K 31/4188; A61K 31/4406; A61K 31/4415; A61K 31/51; A61K 31/519; A61K 31/525; A61K 31/714; A61K 35/60; A61P 13/12; A61P 21/00

(57) ABSTRACT

A composition comprises medium-chain triglycerides (MCT) and at least one ingredient selected from the group consisting of: Fish oil, L-arginine, Vitamin E, Vitamin C, Vitamin B3, Vitamin B5, Vitamin B6, Vitamin B1, Vitamin B2, Folic acid, Biotin, Vitamin B12, and is formulated for use in preventing and/or treating a disease or condition associated with muscle decline in a subject in need thereof. Preferably the subject in need is an individual at risk of or suffering from a renal failure, a renal dysfunction or/and a renal disease, such as chronic kidney disease, protein-energy wasting, end-stage renal disease, kidney failure due to hospitalization in the intensive care unit, acute kidney injury, and the muscle decline is selected from muscle wasting, muscle loss due to kidney failure or dysfunction, muscle decline related to chronic kidney disease, muscle loss due to hospitalization in the intensive care unit during kidney failure, metabolic acidosis during chronic kidney disease, protein-energy wasting or combinations thereof. The method can achieve at least one result that is preventing and/or treating a disease or condition associated with muscle decline in a subject at risk of or suffering from a renal failure, a renal dysfunction or/and a renal disease. The composition and the method may be used for improving the level of at least one amino acid.

The present disclosure generally relates to compositions and methods for preventing and/or treating a disease or condition associated with muscle decline, in particular in a subject at risk of or suffering from a renal failure, a renal dysfunction or/and a renal disease, such as chronic kidney disease, protein-energy wasting, end-stage renal disease, kidney failure due to hospitalization in the intensive care unit, acute kidney injury.

### BACKGROUND OF THE INVENTION

Renal failures, dysfunction or/and diseases, such as chronic kidney disease (CKD), protein-energy wasting, end-stage renal disease (ESRD), kidney failure due to hospitalization in the intensive care unit (ICU), acute kidney injury (AKI), may lead to the constant excessive loss of some amino acids (AA).

The amino acids are structural protein unit molecules of the muscles and their constant loss and/or insufficient amount results in high protein degradation and low protein synthesis, leading to decrease in muscle mass and to muscle wasting.

Thus, muscle decline is a common complication of renal conditions and/or diseases, characterized by the loss of muscle mass, strength and function, which significantly increases the risk of morbidity and mortality in this population.

While nutrient recommendations are typically targeted at overall improvement of muscle declines, no data exists for preventing and/or treating of muscle decline conditions or diseases associated with renal failures, dysfunction or diseases.

Thus, there is a need to identify nutrients necessary to prevent and/or treat muscle decline conditions or diseases associated with renal failures, dysfunction or diseases.

Further, there is a need to provide nutrient compositions and methods for preventing and/or treating a disease or condition associated with muscle decline, in particular in a subject at risk of or suffering from a renal failure, a renal dysfunction or/and a renal disease, such as CKD, protein-energy wasting, ESRD, kidney failure due to hospitalization in the ICU, AKI.

### OBJECT OF THE INVENTION

The aim of the present invention is to provide a method and an optimal nutrient composition having specific ingredients to prevent and/or treat muscle decline conditions or diseases associated with renal failures, dysfunction or diseases, in particular such as CKD, protein-energy wasting, ESRD, kidney failure due to hospitalization in the ICU, AKI.

### SUMMARY OF THE INVENTION

The preclinical study disclosed herein demonstrates that intake of nutrient composition according to the present invention prevents loss of crucial amino acids for muscle proteinogenesis, and thus prevents decrease in muscle mass and muscle wasting in subjects with renal failures, dysfunction or diseases.

Benefits from this improvement include prevention and/or treatment of muscle decline conditions or diseases associated with renal conditions or diseases.

Accordingly, the present disclosure generally relates to novel compositions and methods for preventing and/or treating a disease or condition associated with muscle decline, in particular in a subject at risk of or suffering from a renal failure, a renal dysfunction or/and a renal disease, such as CKD, protein-energy wasting, ESRD, kidney failure due to hospitalization in the ICU, AKI.

Additional features and advantages are described herein and will be apparent from the following Figures and Detailed Description.

### BRIEF DESCRIPTION OF THE FIGURES

FIG. 1a is a graph showing results from the experimental example disclosed herein, demonstrating reduced urinary leucine levels when nutritional composition is given.

FIG. 1b is a graph showing results from the experimental example disclosed herein, demonstrating the absence of significant changes in plasma leucine levels when nutritional composition is given.

FIG. 2a is a graph showing results from the experimental example disclosed herein, demonstrating reduced urinary lysine levels when nutritional composition is given.

FIG. 2b is a graph showing results from the experimental example disclosed herein, demonstrating the absence of significant changes in plasma lysine levels when nutritional composition is given.

FIG. 3a is a graph showing results from the experimental example disclosed herein, demonstrating increased urinary arginine levels when nutritional composition is given.

FIG. 3b is a graph showing results from the experimental example disclosed herein, demonstrating increased plasma arginine levels when nutritional composition is given.

FIG. 4a is a graph showing results from the experimental example disclosed herein, demonstrating reduced urinary methionine levels when nutritional composition is given.

FIG. 4b is a graph showing results from the experimental example disclosed herein, demonstrating decrease of plasma methionine levels when nutritional composition is given.

FIG. 5 is a graph showing results from the experimental example disclosed herein, demonstrating decrease of urinary glutamine levels when nutritional composition is given.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

Within the context of the present invention, the expression “nutritional composition(s)” refers to composition(s) which nourishes a subject. This nutritional composition is usually to be taken enterally, orally, parenterally or intravenously. Preferably, a nutritional composition is for oral use.

Moreover, “nutritional composition(s)” may refer to liquids, powders, gels, pastes, solids, concentrates, suspensions, or ready-to-use forms of enteral formulas, oral formulas, formulas for infants, formulas for children, formulas for adults, porridges and/or cereals, food products, food compositions, baby food, pet food. The compositions of the present disclosure, including the many embodiments described herein, can comprise, consist of, or consist essentially of the essential elements and limitations described herein, as well as any additional or optional ingredients, components, or limitations described herein or otherwise useful in a diet. Food products according to the present invention include, but are not limited to, breads, cakes, cookies, crackers, extruded snacks, potato products, rice products, corn products, wheat products, dairy products, yogurt, confectionery, hard candy, gummy candies, nutrition bar, breakfast cereal or beverage, including a plant-based drink such as a juice, a smoothie, soy milk, rice milk, or almond milk.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

An “individual” is a mammal, preferably a human, a farm animal, a pet. In particular the term “farm animal” may include but is not limited to a horse (e.g., a pet or horse undergoing medical treatment), or cattle or poultry (e.g., cattle or poultry being used in agriculture). In particular the term “pet” means any animal which could benefit from or enjoy the compositions provided by the present disclosure. For example, the pet can be an avian, bovine, canine, equine, feline, hircine, lupine, murine, ovine, or porcine animal, but the pet can be any suitable animal.

A “medium-chain triglyceride(s)” or “MCT” is/are a lipid/lipids in which three fatty acids are bound by ester linkages to a glycerol backbone, and at least two and preferably all three of the fatty acids are each between six and twelve carbons in length. Accordingly, a medium chain fatty acid of MCT may be selected from hexanoate (caproic acid), heptanoate (enanthic acid), octanoate (caprylic acid), nonanoate (pelargonic acid), decanoate (capric acid), undecanoate (undecylic acid) or dodecanoate (lauric acid).

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified. A composition or dosage unit “consisting essentially of” contains at least 50 wt. % of the referenced components, preferably at least 75 wt. % of the referenced components, more preferably at least 85 wt. % of the referenced components, most preferably at least 95 wt. % of the referenced components.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “muscle decline and/or a kidney dysfunction” should be interpreted as “muscle decline” or “a kidney dysfunction” or “both muscle decline and a kidney dysfunction”.

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Chronic kidney disease (CKD)” encompasses the presence of kidney damage (i.e., albuminuria) or decreased kidney function (i.e., GFR <60 mL/min per 1.73 m²) for 3 months or more, irrespective of clinical diagnosis. CKD is classified into five stages on the basis of GFR: more than 90 mL/min per 1.73 m² (stage 1), 60-89 mL/min per 1.73 m² (stage 2), 30-59 mL/min per 1.73 m² (stage 3), 15-29 mL/min per 1.73 m² (stage 4), and less than 15 mL/min per 1.73 m² (stage 5). Early stage of chronic kidney disease encompasses chronic kidney disease in stages 2 and 3 on the basis of GFR: 60-89 mL/min per 1.73 m² (stage 2), 30-59 mL/min per 1.73 m² (stage 3). Preferably, “early stage of chronic kidney disease” encompasses chronic kidney disease in stage 2 on the basis of GFR: 60-89 mL/min per 1.73 m². Late stage of chronic kidney disease encompasses chronic kidney disease in stages 4 and 5 on the basis of GFR: 15-29 mL/min per 1.73 m² (stage 4), and less than 15 mL/min per 1.73 m² (stage 5). Preferably, late stage of chronic kidney disease encompasses chronic kidney disease in stage 4 on the basis of GFR: 15-29 mL/min per 1.73 m².

“End-stage renal disease (ESRD)” encompasses the condition of individuals with CKD, who require a kidney replacement therapy. Preferably, ESRD encompasses chronic kidney disease in stage 5 on the basis of GFR: less than 15 mL/min per 1.73 m².

“Muscle wasting” encompasses a prolonged catabolic state, where muscle protein breakdown exceeds the rate of protein synthesis.

“Protein-energy wasting” relates to a loss of body protein mass and fuel reserves in a subject due to a maladaptive metabolic state. The maladaptive metabolic state includes nonspecific inflammatory processes, transient, intercurrent catabolic illnesses; nutrient losses into dialysate, acidemia, endocrine disorders such as resistance to insulin, growth hormone, and insulin-like growth factor-1, hyperglucagonemia, hyperparathyroidism, and loss of blood into the hemodialyzer, into feces or by blood drawing.

As used herein, an “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects of the composition disclosed herein.

As used herein, the “RPB+” blend is defined in Table 1 below

TABLE 1
RPB+ blend (mg/kg/day)

| | RPB+ |
|---|---|
| Fish oil | 2000 |
| L-Arginine | 2000 |
| Vitamin E acetate, 50% | 100 |
| Vitamin C | 15 |
| Niacin B3 | 4.5 |
| Vitamin B5 | 3.5 |
| Vitamin B6 | 2.4 |
| Thiamine B1 | 1.5 |
| Riboflavin B2 | 0.9 |
| Folic acid | 0.3 |
| Biotin | 0.08 |
| Vitamin B12, 1% | 2.25 |
| MCT | 2500 |

### Embodiments

Amino acid (AA) residues form the second-largest component with only water being the largest of human body tissues, in particular muscles.

In the subjects having renal failures or/and renal diseases, such as chronic kidney disease, protein-energy wasting, end-stage renal disease, kidney failure due to hospitalization in the intensive care unit, acute kidney injury, the high levels of specific AA are revealed in urine, meaning their significant and constant loss by human organism that, as a consequence, leads to failure to their incorporation in muscle or failure in muscle protein synthesis, resulting in muscle decline such as decrease in muscle mass and muscle wasting.

One of the most common diseases leading to muscle declines is CKD. CKD is a gradual and progressive loss of the ability of the kidneys to excrete wastes, concentrate urine, reabsorb proteins and AA, and conserve electrolytes. Unlike AKI with its abrupt but reversible kidney function, the kidney functions in CKD progress and deteriorate irreversibly towards ESRD. CKD arises from many heterogeneous disease pathways that alter the function and structure of the kidney irreversibly, over months or years.

Diabetes and hypertension are the main causes of CKD. Therefore, the investigations were associated with the diabetic subjects.

The inventors surprisingly identified specific nutrients that decrease urine levels of specific AA, such as leucine, lysine, methionine and glutamine, and increase of arginine. Considering that these AA cannot be synthesized by a subject's body and may be obtained only with the supplementation, it is crucial to keep their levels in blood and prevent the body from their loss with kidney dysfunction and/or disease. The decrease of the mentioned AA in urine means prevention of their loss by the human body and keeping their levels sufficient for muscle proteinogenesis, and thus, prevents and/or treats disease or condition associated with muscle decline.

Thus, the invention in the first embodiment relates to a nutritional composition for use in preventing and/or treating a disease or condition associated with muscle decline in a subject in need thereof, comprising medium-chain triglycerides (MCT) and at least one ingredient selected from the group consisting of:
Fish oil,
L-arginine,
Vitamin E,
Vitamin C,
Vitamin B3,
Vitamin B5,
Vitamin B6,
Vitamin B1,
Vitamin B2,
Folic acid,
Biotin,
Vitamin B12.

The MCT may be prepared by any known process, such as direct esterification, rearrangement, fractionation and/or transesterification. The MCT may be synthesised by, for example, esterification of the medium chain fatty acids with glycerol or may be prepared from a source of vegetable oil, such as coconut oil, through a rearrangement process. The chain length and distribution thereof may vary depending on the source oil. The medium-chain fatty acids of the MCT can be selected from the group consisting of hexanoate (caproic acid), heptanoate (enanthic acid), octanoate (caprylic acid), nonanoate (pelargonic acid), decanoate (capric acid), undecanoate (undecylic acid) or dodecanoate (lauric acid), preferably of caprylic acid, capric acid, or a mixture thereof.

Fish oil generally comprises 5 wt. % or more, preferably 10 wt. % or more of DHA.

L-arginine may be naturally occurring or synthesized.

Vitamin E may be incorporated in the composition in the form of alpha-tocopherol, alpha-tocopherol acetate, alpha-tocopherol succinate, alpha-tocopherol nicotinate, a-tocopherol.

Vitamin C may be incorporated in the composition in the form of ascorbic acid.

Vitamin B3 may be incorporated in the composition of the invention as such or in the form of niacin, nicotinic acid, nicotinamide, niacinamide, nicotinamide adenine dinucleotide, NAD, nicotinic acid mononucleotide, NicMN, pyridine-3-carboxylic acid.

Vitamin B5 may be incorporated in the composition of the invention as such or in the form of pantothenic acid—pantothenate, panthenol.

Vitamin B6 may be incorporated in the composition of the invention as such or in the form of pyridoxine, pyridoxal, pyridoxamine, pyridoxine hydrochloride.

Vitamin B1 may be incorporated in the composition of the invention as such or in the form of thiamin, thiamin pyrophosphate, TPP, thiamin triphosphate, TTP, thiamin hydrochloride, thiamin mononitrate.

Vitamin B2 may be incorporated in the composition of the invention as such or in the form of riboflavin, flavin mononucleotide, FMN, flavin adenine dinucleotide, FAD, lactoflavin, ovoflavin.

Vitamin B12 may be incorporated in the composition of the invention as such or in the form of a physiologically acceptable salt thereof or mixtures thereof, or via any source comprising Vitamin B12. In particular Vitamin B12 may be incorporated into the composition in its pure form, as cyanocobalamin, hydroxocobalamin, and any combination thereof.

In one embodiment, the present invention relates to the nutritional composition comprising medium-chain triglycerides (MCT) in a daily amount ranging from about 1000 to about 20000 mg per kg of weight of the subject and at least one ingredient selected from the group consisting of
Fish oil in a daily amount ranging from about 100 to about 5000 mg per kg of weight of the subject,
L-arginine in a daily amount ranging from about 100 to about 50000 mg per kg of weight of the subject,
Vitamin E in a daily amount ranging from about 1 to about 80000 mg per kg of weight of the subject,
Vitamin C in a daily amount ranging from about 0.1 to about 2000 mg per kg of weight of the subject,
Vitamin B3 in a daily amount ranging from about 0.2 to about 10 mg per kg of weight of the subject,
Vitamin B5 in a daily amount ranging from about 0.08 to about 500 mg per kg of weight of the subject,
Vitamin B6 in a daily amount ranging from about 0.0.3 to about 50 mg per kg of weight of the subject,
Vitamin B1 in a daily amount ranging from about 0.03 about 5 mg per kg of weight of the subject,
Vitamin B2 in a daily amount ranging from about 0.03 to about 5 mg per kg of weight of the subject,
Folic acid in a daily amount ranging from about 0.01 to about 5 mg per kg of weight of the subject,
Biotin in a daily amount ranging from about 0.01 to about 0.1 mg per kg of weight of the subject,
Vitamin B12 in a daily amount ranging from about 0.001 to about 5 mg per kg of weight of the subject.

In one embodiment, the present invention relates to the nutritional composition for administration to human subject, comprising medium-chain triglycerides (MCT) in a daily amount ranging from about 1000 to about 20000 mg per kg of weight of the subject and at least one ingredient selected from the group consisting of
Fish oil in a daily amount ranging from about 100 to about 5000 mg per kg of weight of the subject, preferably in an amount ranging from about 200 to about 3000 mg per kg of weight of the subject,
L-arginine in a daily amount ranging from about 1000 to about 50000 mg per kg of weight of the subject, preferably in an amount ranging from about 3000 to about 30000 mg per kg of weight of the subject,
Vitamin E in a daily amount ranging from about 100 to about 80000 mg per kg of weight of the subject, preferably in an amount ranging from about 500 to about 50000 mg per kg of weight of the subject,
Vitamin C in a daily amount ranging from about 100 to about 2000 mg per kg of weight of the subject, preferably in an amount ranging from about 500 to about 1000 mg per kg of weight of the subject,
Vitamin B3 in a daily amount ranging from about 1 to about 10 mg per kg of weight of the subject, preferably in an amount ranging from about 2 to about 6 mg per kg of weight of the subject,
Vitamin B5 in a daily amount ranging from about 50 to about 500 mg per kg of weight of the subject, preferably in an amount ranging from about 100 to about 200 mg per kg of weight of the subject,
Vitamin B6 in a daily amount ranging from about 0.5 to about 50 mg per kg of weight of the subject, preferably in an amount ranging from about 1 to about 25 mg per kg of weight of the subject,
Vitamin B1 in a daily amount ranging from about 0.2 to about 5 mg per kg of weight of the subject, preferably in an amount ranging from about 0.5 to about 2 mg per kg of weight of the subject,
Vitamin B2 in a daily amount ranging from about 0.2 to about 5 mg per kg of weight of the subject, preferably in an amount ranging from about 0.5 to about 2 mg per kg of weight of the subject,
Folic acid in a daily amount ranging from about 0.2 to about 5 mg per kg of weight of the subject, preferably in an amount ranging from about 0.5 to about 1 mg per kg of weight of the subject,
Biotin in a daily amount ranging from about 0.01 to about 0.1 mg per kg of weight of the subject, preferably in an amount ranging from about 0.02 to about 0.04 mg per kg of weight of the subject,
Vitamin B12 in a daily amount ranging from about 0.5 to about 5 mg per kg of weight of the subject. preferably in an amount ranging from about 1.5 to about 2 mg per kg of weight of the subject.

In a preferred embodiment, the nutritional composition administered to a pet animal, comprises medium-chain triglycerides (MCT) in a daily amount ranging from about 100 to about 2000 mg per kg of weight of the pet animal and at least two ingredients selected from the group consisting of:
Fish oil in a daily amount ranging from about 100 to about 5000 mg per kg of weight of the subject, preferably in an amount ranging from about 200 to about 3000 mg per kg of weight of the pet animal,
L-arginine in a daily amount ranging from about 100 to about 600 mg per kg of weight of the pet animal, preferably 375 mg/kg BW for a dog and 575 mg/kg BW for a cat.
Vitamin E in a daily amount ranging from about 1 to about 100 mg per kg of weight of the the pet animal, preferably 8 mg/kg BW for a dog and 14 mg/kg BW for a cat. 8.27 mg/kg BW.
Vitamin C in a daily amount ranging from about 0.1 to about 10 mg per kg of weight of the pet animal, preferably 1.3 mg/kg BW for a dog and 2 mg/kg BW for a cat.
Vitamin B3 in a daily amount ranging from about 0.2 to about 10 mg per kg of weight of the pet animal, preferably 1.5 mg/kg BW for a dog,
Vitamin B5 in a daily amount ranging from about 0.08 to about 20 mg per kg of weight of the pet animal, preferably 0.5 mg/kg BW for a dog, and 1.4 mg/kg BW for a cat,
Vitamin B6 in a daily amount ranging from about 0.0.3 to about 10 mg per kg of weight of the pet animal, preferably 0.2 mg/kg BW for a dog, and 0.5 mg/kg BW for a cat,
Vitamin B1 in a daily amount ranging from about 0.03 about 5 mg per kg of weight of the pet animal, preferably 0.3 mg/kg BW for a dog, and 1.4 mg/kg BW for a cat,
Vitamin B2 in a daily amount ranging from about 0.03 to about 5 mg per kg of weight of the pet animal, preferably 0.2 mg/kg BW for a dog, and 0.8 mg/kg BW for a cat,
Folic acid in a daily amount ranging from about 0.01 to about 5 mg per kg of weight of the pet animal, preferably 0.06 mg/kg BW for a dog, and 0.11 mg/kg BW for a cat,
Biotin in a daily amount ranging from about 0.01 to about 0.1 mg per kg of weight of the pet animal
Vitamin B12 in a daily amount ranging from about 0.001 to about 5 mg per kg of weight of the pet animal, preferably 0.015 mg/kg BW for a dog, and 0.002 mg/kg BW for a cat.

In one embodiment, the present invention relates to the nutritional composition as described above wherein the subject is an individual at risk of or suffering from a renal failure, a renal dysfunction or/and a renal disease.

In one embodiment, the present invention relates to the nutritional composition as described above wherein the individual is a human, a farm animal or a pet.

In one embodiment, the present invention relates to the nutritional composition as described above wherein the muscle decline is related to a renal failure, a renal dysfunction or/and a renal disease.

In one embodiment, the present invention relates to the nutritional composition as described above wherein the renal failure, the renal dysfunction or/and the renal disease is selected from the list of chronic kidney disease, protein-energy wasting, end-stage renal disease, kidney failure due to hospitalization in the intensive care unit, acute kidney injury.

In one embodiment, the present invention relates to the nutritional composition as described above wherein the disease or condition associated with muscle decline is selected from the list consisting of muscle wasting, muscle loss due to kidney failure or dysfunction, muscle decline related to chronic kidney disease, muscle loss due to hospitalization in the intensive care unit during kidney failure, metabolic acidosis during chronic kidney disease, protein-energy wasting or combinations thereof.

In one embodiment, the present invention relates to the nutritional composition as described above comprising at least three ingredients selected from said group.

In one embodiment, the present invention relates to the nutritional composition as described above comprising at least four ingredients selected from said group.

In one embodiment, the present invention relates to the nutritional composition as described above comprising at least five ingredients selected from said group.

In one embodiment, the present invention relates to the nutritional composition as described above comprising at least six ingredients selected from said group.

In one embodiment, the present invention relates to the nutritional composition as described above comprising at least seven ingredients selected from said group.

In one embodiment, the present invention relates to the nutritional composition as described above comprising at least eight ingredients selected from said group.

In one embodiment, the present invention relates to the nutritional composition as described above comprising at least nine ingredients selected from said group.

In one embodiment, the present invention relates to the nutritional composition as described above comprising at least ten ingredients selected from said group.

In one embodiment, the present invention relates to the nutritional composition as described above comprising at least eleven ingredients selected from said group.

In one embodiment, the present invention relates to the nutritional composition as described above comprising at least twelve ingredients selected from said group.

In one embodiment, the present invention relates to the nutritional composition as described above for use in improving the level of at least one amino acid.

In one embodiment, the present invention relates to the nutritional composition as described above wherein at least one amino acid is selected from the group consisting of leucine, lysine, arginine, methionine and glutamine.

The nutritional composition as disclosed herein can use any of a variety of formulations for administration.

In preferable embodiment, the formulation of administration is oral in various kinds of formulas, food and food products as mentioned above.

The compositions of the present disclosure may comprise any additional or optional ingredients, components, or limitations described herein or otherwise useful in a diet.

The person skilled in the art would identify appropriate amounts of the above-mentioned nutrients, metabolic precursors or metabolites thereof to achieve in the nutritional composition after administration their highest permitted levels.

In one embodiment, the present invention relates to a method of preventing and/or treating a disease or condition associated with muscle decline comprising administering to a subject in need thereof an effective amount of the nutritional composition as described above.

In one embodiment, the present invention relates to the method as described above wherein the subject is an individual at risk of or suffering from a renal failure, a renal dysfunction or/and a renal disease.

In one embodiment, the present invention relates to the method as described above wherein the muscle decline is related to a renal failure, a renal dysfunction or/and a renal disease.

In one embodiment, the present invention relates to the method as described above wherein the renal failure, the renal dysfunction or/and the renal disease is selected from the list of chronic kidney disease, protein-energy wasting, end-stage renal disease, kidney failure due to hospitalization in the intensive care unit, acute kidney injury.

In one embodiment, the present invention relates to the method as described above wherein the disease or condition associated with muscle decline is selected from the list consisting of muscle wasting, muscle loss due to kidney failure or dysfunction, muscle decline related to chronic kidney disease, muscle loss due to hospitalization in the intensive care unit during kidney failure, metabolic acidosis during chronic kidney disease, protein-energy wasting or combinations thereof.

In one embodiment, the present invention relates to the method as described above of improving the level of at least one amino acid.

In one embodiment, the present invention relates to the method as described above wherein at least one amino acid is selected from the group consisting of leucine, lysine, arginine, methionine, glutamine.

### Health Effect

The health effect of the present invention may be preventive, for example, preventing proteinogenesis failure and as a consequence muscle decline conditions, or curative, for example, treating a disease or condition associated with muscle decline, such as muscle wasting, muscle loss due to kidney failure or dysfunction, muscle decline related to chronic kidney disease, muscle loss due to hospitalization in the intensive care unit during kidney failure, metabolic acidosis during chronic kidney disease, protein-energy wasting or combinations thereof, in subjects at risk of or suffering from renal conditions.

The composition of the present invention comprises specific ingredients that impact on improving levels of amino acids in the human body which are key nutrients for muscle repair and growth, transporting energy to the muscles in the body and inhibiting the reduction of muscle protein at the same time.

These beneficial effects are specifically important in subjects having renal failures or/and renal diseases, such as chronic kidney disease, protein-energy wasting, end-stage renal disease, kidney failure due to hospitalization in the intensive care unit, acute kidney injury, that experience the high and constant loss of the AA, which could be revealed in urine, leading to the failure in muscle protein synthesis, and, as the consequence, to muscle declines such as decrease in muscle mass and muscle wasting.

Other advantages of the present invention are improving the endurance and/or efficiency of a muscle, the muscle fiber size, and reducing muscle atrophy.

Thus, the benefit of the present invention is maintaining proteinogenesis, prevention of the lean of mass of a muscle and muscle wasting, maintaining healthy muscle mass and treatment of muscle declines and diseases at subjects with renal conditions.

### EXAMPLES

The following non-limiting example generally illustrates the concepts underlying the embodiments disclosed herein.

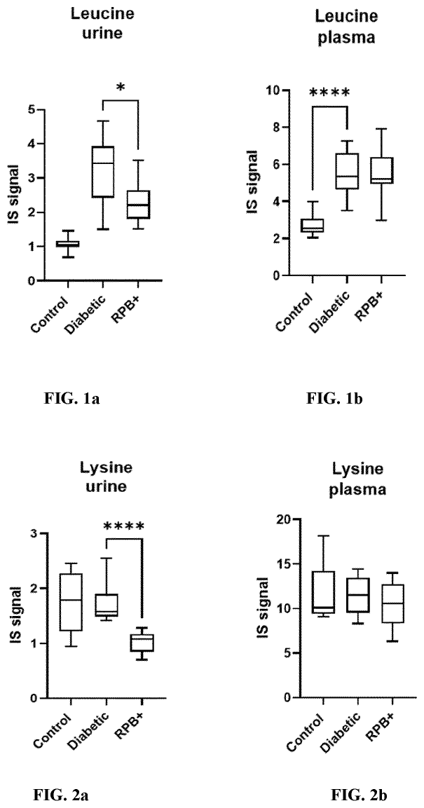

FIG. 1a shows the urinary leucine level measured in mice after 8 weeks treatment with the disclosed nutritional composition-hereinafter Renal Protective Blend plus (RPB+) compared to a vehicle group (Diabetic, db/db+V group) and a control group (Healthy, db/m+V group) after 8 weeks treatment with 100 μl of 0.5% carboxymethylcellulose. The urinary leucine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH₄OH), pH ˜9.3, and solvent B was acetonitrile (ACN). The eluting metabolites, leucine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed a significant decrease of urinary leucine level in mice treated with RPB+ compared to diabetic control group, translated to a better kidney reabsorption through proximal tubules cells to maintain circulating dose of leucine for beneficial effect on muscle.

FIG. 1b shows the plasma leucine level measured in mice after 8 weeks treatment with Renal Protective Blend plus (RPB+) compared to a vehicle group (Diabetic, db/db+V group) and a control group (Healthy, db/m+V group) after 8 weeks treatment with 100 μl of 0.5% carboxymethylcellulose. The plasma leucine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH₄OH), pH ˜9.3, and solvent B was acetonitrile (ACN). The eluting metabolites, leucine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed no differences between mice treated with RPB+ and diabetic control group.

FIG. 2a shows the urinary lysine level measured in mice after 8 weeks treatment with Renal Protective Blend plus (RPB+) compared to a vehicle group (Diabetic, db/db+V group) and a control group (Healthy, db/m+V group) after 8 weeks treatment with 100 μl of 0.5% carboxymethylcellulose. The urinary lysine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH₄OH), pH ˜9.3, and solvent B was acetonitrile (ACN). The eluting metabolites, lysine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed a significant decrease of urinary lysine level in mice treated with RPB+ compared to diabetic control group, translated to a better kidney reabsorption through proximal tubules cells to maintain circulating dose of lysine for beneficial effect on muscle.

FIG. 2b shows the plasma lysine level measured in mice after 8 weeks treatment with Renal Protective Blend plus (RPB+) compared to a vehicle group (Diabetic, db/db+V group) and a control group (Healthy, db/m+V group) after 8 weeks treatment with 100 μl of 0.5% carboxymethylcellulose. The plasma lysine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH₄OH), pH ˜9.3, and solvent B was acetonitrile (ACN). The eluting metabolites, lysine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed no differences between mice treated with RPB+ and diabetic control group.

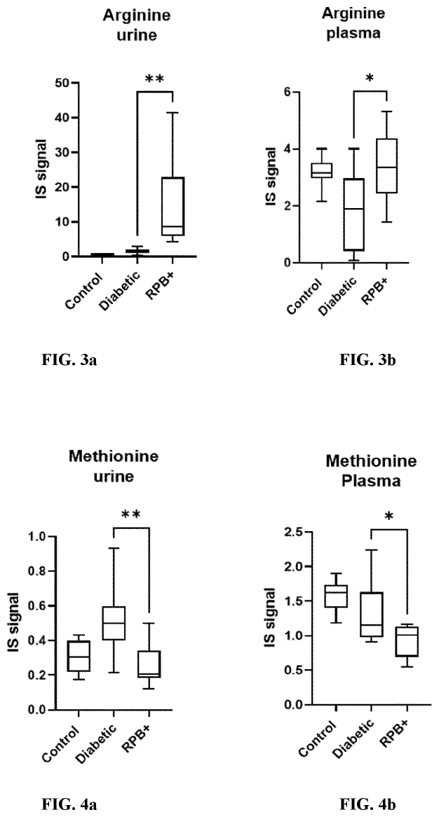

FIG. 3a shows the urinary arginine level measured in mice after 8 weeks treatment with Renal Protective Blend plus (RPB+) compared to a vehicle group (Diabetic, db/db+V group) and a control group (Healthy, db/m+V group) after 8 weeks treatment with 100 μl of 0.5% carboxymethylcellulose. The urinary arginine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH₄OH), pH ˜9.3, and solvent B was acetonitrile (ACN). The eluting metabolites, arginine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed a significant increase of urinary arginine level in mice treated with RPB+ compared to diabetic control group due to arginine supplementation present in the blend.

FIG. 3b shows the plasma arginine level measured in mice after 8 weeks treatment with Renal Protective Blend plus (RPB+) compared to a vehicle group (Diabetic, db/db+V group) and a control group (Healthy, db/m+V group) after 8 weeks treatment with 100 μl of 0.5% carboxymethylcellulose. The plasma arginine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH4OH), pH ˜9.3, and solvent B was acetonitrile (ACN). The eluting metabolites, arginine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed a significant increase of plasma arginine in mice treated with RPB+ compared to diabetic control group. The RPB supplementation rescued arginine depletion present in diabetic condition.

FIG. 4a shows the urinary methionine level measured in mice after 8 weeks treatment with Renal Protective Blend (RPB+) compared to a vehicle group (Diabetic, db/db+V group) and a control group (Healthy, db/m+V group) after 8 weeks treatment with 100 μl of 0.5% carboxymethylcellulose. The urinary methionine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH4OH), pH ˜9.3, and solvent B was acetonitrile (ACN). The eluting metabolites, methionine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed a significant decrease of urinary methionine level in mice treated with RPB+ compared to diabetic control group, translated to a better kidney reabsorption through proximal tubules cells to maintain circulating dose of methionine for beneficial effect on muscle.

FIG. 4b shows the plasma methionine level measured in mice after 8 weeks treatment with Renal Protective Blend (RPB+) compared to a vehicle group (Diabetic, db/db+V group) and a control group (Healthy, db/m+V group) after 8 weeks treatment with 100 μl of 0.5% carboxymethylcellulose. The plasma methionine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH4OH), pH ˜9.3, and solvent B was acetonitrile (ACN). The eluting metabolites, methionine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed a significant decrease of plasma methionine level in mice treated with RPB+ compared to diabetic control group.

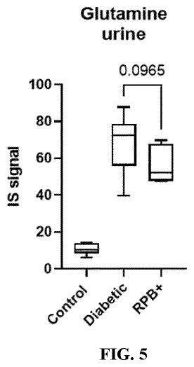

FIG. 5 shows the urinary glutamine level measured in mice after 8 weeks treatment with Renal Protective Blend (RPB) compared to a vehicle group (Diabetic, db/db+V group) and a control group (Healthy, db/m+V group) after 8 weeks treatment with 100 μl of 0.5% carboxymethylcellulose. The urinary glutamine level was determined using a liquid-liquid extraction with 13C-yeast as internal standards. 20 μL of urine were directly extracted in 1300 μL cold methanol:water:chloroform (5:3:5 (v/v)). All samples were agitated for 10 minutes at 1500 rpm and 4° C. in a shaker (Thermomixer C, Eppendorf), followed by centrifugation for 10 minutes and 15,000 rpm at 4° C. Two phases were obtained after the centrifugation step: an upper phase containing the polar metabolites and a lower phase containing apolar metabolites. In addition, a protein layer remained in the middle between the two phases. The upper phase was dried overnight in a vacuum centrifuge at 4° C. and 5 mbar, and was dissolved in 60 μL 60% (v/v) acetonitrile:water prior to analysis. The protein layer was quantified with a bicinchoninic acid (BCA) assay (ThermoFisher Scientific) and used for later normalization of the metabolite concentrations. Two microliters of each sample were injected into a hydrophilic interaction chromatography (HILIC) analytical column. The separation was achieved by applying a linear solvent gradient. As mobile phase, solvent A was H2O with 10 mM ammonium acetate (NH4Ac) and 0.04% (v/v) ammonium hydroxide (NH4OH), pH ˜9.3, and solvent B was acetonitrile (ACN). The eluting metabolites, glutamine, were analyzed with an orbitrap mass spectrometer (Orbitrap Fusion Lumos Tribrid, Thermo Scientific) equipped with a heated electrospray ionization (H-ESI) source. On-the-fly alternating negative (3 kV) and positive (3.5 kV) ion modes was used for ionization. The software Xcalibur v4.1.31.9 (Thermo Scientific) was used for instrument control, data acquisition and processing.

Data showed a tendency to decrease of urinary glutamine level in mice treated with RPB+ compared to diabetic control group.

This preclinical study demonstrates that intake of the nutrition composition disclosed herein prevents the loss of crucial amino acids of muscle proteins and thus, prevents and/or treats a disease or condition associated with muscle decline, in particular in a subject at risk of or suffering from a renal failure, a renal dysfunction or/and a renal disease, such as CKD, protein-energy wasting, ESRF, kidney failure due to hospitalization in the ICU, AKI.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of preventing and/or treating a disease or condition associated with muscle decline in a subject in need thereof, the method comprising administering to the subject an effective amount of a nutritional composition comprising medium-chain triglycerides (MCT) and at least one ingredient selected from the group consisting of:
Fish oil,
L-arginine,
Vitamin E,
Vitamin C,
Vitamin B3,
Vitamin B5,
Vitamin B6,
Vitamin B1,
Vitamin B2,
Folic acid,
Biotin, and
Vitamin B12.

2. The method of claim 1, wherein the nutritional composition comprises the medium-chain triglycerides (MCT) in a daily amount ranging from 1000 to about 20000 mg per kg of weight of the subject, and the at least one ingredient is selected from the group consisting of:
Fish oil in a daily amount ranging from about 100 to about 5000 mg per kg of weight of the subject,
L-arginine in a daily amount ranging from about 100 to about 50000 mg per kg of weight of the subject,
Vitamin E in a daily amount ranging from about 1 to about 80000 mg per kg of weight of the subject,
Vitamin C in a daily amount ranging from about 0.1 to about 2000 mg per kg of weight of the subject,
Vitamin B3 in a daily amount ranging from about 0.2 to about 10 mg per kg of weight of the subject,
Vitamin B5 in a daily amount ranging from about 0.08 to about 500 mg per kg of weight of the subject,
Vitamin B6 in a daily amount ranging from about 0.03 to about 50 mg per kg of weight of the subject,
Vitamin B1 in a daily amount ranging from about 0.03 about 5 mg per kg of weight of the subject,
Vitamin B2 in a daily amount ranging from about 0.03 to about 5 mg per kg of weight of the subject,
Folic acid in a daily amount ranging from about 0.01 to about 5 mg per kg of weight of the subject,
Biotin in a daily amount ranging from about 0.01 to about 0.1 mg per kg of weight of the subject, and
Vitamin B12 in a daily amount ranging from about 0.001 to about 5 mg per kg of weight of the subject.

3. The method of claim 1, wherein the subject is an individual at risk of or suffering from a renal failure, a renal dysfunction or/and a renal disease.

4. The method of claim 1, wherein the individual is a human, a farm animal or a pet.

5. The method of claim 1, wherein the muscle decline is related to a renal failure, a renal dysfunction or/and a renal disease.

6. The method of claim 1, wherein the renal failure, the renal dysfunction or/and the renal disease is selected from the list of chronic kidney disease, protein-energy wasting, end-stage renal disease, kidney failure due to hospitalization in the intensive care unit, acute kidney injury.

7. The method of claim 1, wherein the disease or condition associated with muscle decline is selected from the list consisting of muscle wasting, muscle loss due to kidney failure or dysfunction, muscle decline related to chronic kidney disease, muscle loss due to hospitalization in the intensive care unit during kidney failure, metabolic acidosis during chronic kidney disease, protein-energy wasting or combinations thereof.

8. The method of claim 1, wherein the nutritional composition comprises at least three ingredients selected from the group.

9. The method of claim 1, wherein the nutritional composition improves a level of at least one amino acid in the subject.

10. The method of claim 9, wherein the at least one amino acid is selected from the group consisting of leucine, lysine, arginine, methionine, and glutamine.

11-17. (canceled)

18. The method of claim 1, wherein the nutritional composition comprises at least four ingredients selected from the group.

19. The method of claim 1, wherein the nutritional composition comprises at least five ingredients selected from the group.

20. The method of claim 1, wherein the nutritional composition comprises at least six ingredients selected from the group.

21. The method of claim 1, wherein the nutritional composition comprises at least seven ingredients selected from the group.

22. The method of claim 1, wherein the nutritional composition comprises at least eight ingredients selected from the group.

23. The method of claim 1, wherein the nutritional composition comprises at least nine ingredients selected from the group.

24. The method of claim 1, wherein the nutritional composition comprises at least ten ingredients selected from the group.

25. The method of claim 1, wherein the nutritional composition comprises at least eleven ingredients selected from the group.

26. The method of claim 1, wherein the nutritional composition comprises at least twelve ingredients selected from the group.
